(19) United States
(12) Patent Application Publication
(10) Pub. No.: US 2010/0062479 A1
(43) Pub. Date: Mar. 11, 2010

(54) METHOD AND DEVICE

(76) Inventors: Gesa RASCHER-EGGSTEIN, Kessin (DE); Thomas Knott, Rostock (DE)

Correspondence Address: MORRISON & FOERSTER LLP, 12531 HIGH BLUFF DRIVE, SUITE 100, SAN DIEGO, CA, 92130-2040, US

(21) Appl. No.: 12/553,886

(22) Filed: Sep. 3, 2009

Related U.S. Application Data
(63) Continuation of application No. PCT/EP2008/001664, filed on Mar. 1, 2008.

(30) Foreign Application Priority Data
Mar. 4, 2007 (DE) 10 2007 010 843.7

(51) Int. Cl. C12Q 1/02; C12N 5/02; C12M 1/00

(52) U.S. Cl. 435/29; 435/374; 435/307.1

(57) ABSTRACT

A method of providing suspended cells (10) for carrying out biological assays, comprising the steps of:

a) providing a frozen cell suspension,

b) bringing the frozen cell suspension into direct contact with an excess volume of thawing buffer (12), and

c) incubating the solution of step b) at room temperature in order to thaw the cell suspension.

This application is a continuation of international patent application PCT/EP2008/001664 filed on Mar. 1, 2008, and designating the U.S., which claims priority from German utility patent 10 2007 010 843.7 filed on Mar. 4, 2007. The entire contents of these priority applications are incorporated herein by reference.

The present invention relates to methods and devices by means of which individual biological cells are provided which are to be used in biological assays with high specifications regarding membrane quality.

Many applications in cell biology, medicine and generally in biotechnology, and the biological assays mentioned at the outset require the long-term storage of living biological cells, in particular microorganisms, eukaryotic cells and/or cell lines after they have been extracted or generated so that they can be transported and/or used at a later point in time. During this storage, the metabolic functions in the cell should be stopped in order to find the cell as unchanged as possible after storage and in a manner of speaking to be able to use it just as before storage.

It has long been known that biological cells may be frozen for such purposes and, after suitable storage, thawed again. At the lowest temperatures of up to for example −196° C., the frozen cells may be stored for many weeks, months or years.

In order to ensure a high survival rate of the frozen cells, the storage medium for cryopreservation and the freezing and thawing methods must meet particular specifications. For example, a cryoprotective agent (CPA) such as, for example, dimethyl sulphoxide (DMSO) must be present in the storage medium to prevent the formation of ice crystals, which might damage the cell membrane. Another problem in cryopreservation, besides the formation of ice crystals, is osmolarity stress, because partially thawed or partially frozen storage medium may lead to locally increased salt and/or CPA concentrations and to a washing-out of the CPA from the cells. It must furthermore be taken into consideration that the CPA may be toxic. DMSO, for example, is only not toxic when used at low concentrations and low temperatures.

An early review over methods of freezing living cells and the problems which this entails can be found in P. Mazur (1984), “Freezing of Living Cells: mechanisms and implications,” Am. J. Physiol. 247, pages 125 to 142. Suitable CPAs can be found for example in K. G. Brockband (1995), Principles of Autologous, Allogenic and Cryopreserved Venous Transplantation, Chapter 10, Table 10.1. The content of these publications is by reference expressly a constituent of the present application.

U.S. Pat. No. 5,700,632 by Critser discloses a method in which biological cells are frozen and then again thawed using specific temperature profiles to be calculated with particular formulae, where the CPA is washed out of the thawed cell suspension with the aid of an isotonic cell culture medium and using a specific porous membrane.

WO 2007/009285 discloses a method for the cryopreservation of in-vitro cultivated cells, in which method samples of in each case 2×10E6 cells from different cell lines in 1 ml of a specific storage medium are taken up into cryovials, where they are frozen from room temperature to −80° C. at a controlled freezing rate of 0.5 to 5° C./min and then stored in liquid nitrogen. The cells are then again thawed by firstly liquefying the frozen samples for 3 minutes in a waterbath at 37° C. and then transferring them into a sterile tube containing 10 ml of fresh medium. After 5 minutes centrifugation at 350 g, the supernatant is removed, and the cell pellet is resuspended in 3 ml of fresh medium.

WO 01/78504 A2 discloses a two-step method of thawing cryopreserved cells in which the frozen cells are first warmed in their cryovials in an air bath of less than 30° C. to an intermediate temperature of at least −30° C., and then warmed further in a waterbath of at least 32° C.

US 2003/0039952 A1 describes three different methods for the thawing of frozen cell suspension. In these methods a volume of 25 ml of thawing buffer is added to 25 ml of cell suspension, upon which the cells are thawed and incubated in a growth medium over night at, for example, 37° C. After that the cells are expanded and employed further.

A particular disadvantage of these known methods is that they are complicated, expensive and time consuming, and that the thawed cells frequently must be further cultivated in a cell laboratory before they are actually used.

Furthermore, the known methods frequently have several further disadvantages because they solve the abovementioned problems emerging from freezing and/or thrawing only partially or not at all. This results in the formation of intracellular ice crystals as the result of recrystallization, in osmolarity stress as the result of partially thawed media, with corresponding increased salt concentration, osmolarity stress as the result of washing-out of the CPA from the cells, and toxic effects of the CPA in the thawed state. As a consequence, the survival rate of the frozen cells is frequently unsatisfactory, which is economically disadvantageous taking into consideration the high costs which arise in the provision of many cells and cell lines.

Besides the viability of the thawed cells, it is in particular the cell membrane quality of the thawed cells which plays a decisive role for the applications mentioned at the outset.

The membrane qualities which can be achieved with the known methods are generally sufficient for those assays in which adherent cells are used, or such cells as are generated by seeding thawed suspended cells in a cell culture vessel. The known thawing methods are also sufficient when using suspended cells in assays with low specifications regarding the membrane quality, such as flux assay or fluorescence assay.

In the fields of application mentioned at the outset in which suspended single cells are used, however, the membrane quality must meet high specifications. This is the case for example in the case of automated and manual patch clamping as disclosed for example in EP 1 218 736 A, EP 0 938 674 A or EP 1 311 655 A. The quality of the cell membranes of the cells contacted via the patch clamp technique is decisive for the formation of a gigaseal which can be maintained over a sufficiently long period of time.

Further fields of application with high specifications regarding the membrane quality are flow cytometry and voltage-sensitive membrane dyes.

To date, suspended cells for such purposes are provided by means of continuous cell culture, and only in this manner are the known methods capable of reliably ensuring the membrane quality required. However, this requires a cell laboratory with expensive equipment and suitable know-how.

Finally, the thawed cells must be provided over a certain period of time in a single-cell suspension in order to be used in the assays.

According to the prior art, the suspended cells are maintained either in unstirred or in stirred vessels, which entails specific disadvantages in each case.

Owing to the magnetic stirring devices used, the stirred vessels are not suitable for maintaining thawed cells in single-cell suspension at high vitality and membrane quality in the small volume ranges which are meaningful for the biological assays to be carried out. This is because volumes of less than 20 ml require the magnetic stirrer to be operated at higher rotation rates, owing to the laminar flow situation, which, in turn, leads to frequent cell-damaging contact between magnetic stirrer and cells.

However, unstirred vessels are also not suitable because the individual cells adhere to the walls of the vessel, settle and are damaged by the acidic environment which forms.

In both the cases of vessels with a magnetic stirrer or unstirred vessels, further, adherence between the cells occurs, resulting in the formation of undesired cell accumulations.

In order to be able to use biological cells in many different ways for assays with high specifications regarding the membrane quality, it would therefore be desirable if it were possible to thaw the cells after cryopreserved storage and transport in a simple and membrane-conserving manner and to maintain them in single-cell suspension over a certain period of time.

The object underlying the present invention is therefore to provide methods and devices by means of which biological cells in small volumes can be provided in single-cell suspension and with high membrane quality and kept available over several hours while proceeding in an inexpensive and technically simple manner.

In accordance with the invention, this object is achieved by a method in which a cryopreserved suspension of cells is provided in a storage medium with at least one CPA, which suspension is, for the purpose of thawing, directly brought into contact with a thawing buffer which is approximately at room temperature.

The method according to the invention for providing suspended cells for carrying out biological assays comprises the following steps:

a) providing a frozen cell suspension,

b) bringing the frozen cell suspension into direct contact with an excess volume of thawing buffer, and

c) incubating the solution of step b) at room temperature in order to thaw the cell suspension.

In this manner, the object underlying the invention is achieved entirely.

Surprisingly, the new method is suitable for preparing, rapidly, simply and inexpensively, from a frozen cell suspension a single-cell suspension of high quality while avoiding the above-discussed disadvantages and problems without requiring the effort of a continuous cell culture.

In one embodiment, the thawing buffer is present in at least ten times the volume of the frozen cells. A suitable range for the excess volume of thawing buffer over frozen cell suspension sample is from about 1:10 to about 1:40.

This excess volume ensures high cell viability and good membrane quality by causing a quick thawing process due to the continuity of a steep temperature gradient between the frozen cell suspension pellet and the surrounding thawing buffer throughout the thawing process. This way, also external heating, e.g., by handwarming or incubation in a water bath, potentially exposing the cells to contaminants, is made unnecessary. Additionally, during this thawing process, dissolved buffer components are evenly distributed, avoiding osmotic stress, and the CPA present in the frozen cell suspension sample is quickly washed out and diluted, avoiding CPA toxicity.

In one embodiment, in step b), a frozen cell pellet is immersed directly in the thawing buffer which is present in a thawing vessel at approximately room temperature.

In this embodiment, it is advantageous that the frozen cell pellet is directly contacted by thawing buffer from all sides, which further reduces the time for the thawing process including the time for the even distribution of dissolved substances and the dilution of a CPA. Further, it is beneficial that the thawing process is carried out at room temperature which additionally leads to the reduction of CPA toxicity. Moreover, the liquid surrounding the pellet from all sides absorbs strong vibrations, exerted on the vessel during handling and agitation, this way reducing the risk of recrystallization inside the cells.

In one embodiment, in step a), the frozen cells are provided in an open transport vessel and, in step b), the transport vessel is immersed directly in the thawing buffer which is present in a thawing vessel at approximately room temperature.

The major advantage of this embodiment is that the cell pellet does not have to be manually removed from a transport vessel, reducing the risk of contamination. Further, frozen cell suspension samples can be supplied inside the transport vessel, inside a suitable, sterile, individual package, from which they can, after opening, directly be transferred into the thawing vessel by tilting or inverting the package. In this case, the risk of contaminations is counteracted during both the storage of the frozen cell suspension and the thawing process. Additionally, the beneficial aspects of direct immersion into a thawing buffer remain present. Preferably using thin-walled transport vessels, the immersion into thawing buffer will lead to a quick release of the cell pellet from the transport vessel walls and, this way, ensure an even quicker thawing process.

In one embodiment, the transport vessel is arranged on the inside of a lid for the thawing vessel, which lid is placed on the thawing vessel in step b), which thawing vessel is then tilted in order to bring the thawing buffer into contact with the cells.

The advantage of arranging the transport vessel inside a lid for the thawing vessel, is the reduction of the contamination risk and, moreover, a reduction of the handling time of the cell pellet. This shortened handling time reduces the risk of an untimely thawing of the cell pellet still outside of the thawing buffer.

In one embodiment, the thawing vessel in step c) is shaken manually or mechanically.

The agitation of the vessel by shaking accelerates the thawing process by temperature equalization inside the thawing vessel, further supporting the distribution of dissolved substances and the dilution of CPA by generating a flow in the vessel.

In one embodiment, after step c), the cells are separated from the supernatant by centrifugation, filtering or sedimentation, the supernatant is discarded and the cell pellet is taken up once more in the thawing buffer in a storage vessel.

In this embodiment, it is beneficial that the cells after their resuspension in thawing buffer are alleviated from the toxic effects of the CPA, and that the effects of the CPA whether toxicity dependent or independent do not exert any influence on further experimentation. Additionally, the composition of the medium, with exception of the CPA, remains constant throughout freezing, thawing and subsequent storage.

In one embodiment, the storage vessel is subjected to intermediate storage in a device in which measures which maintain the single cell suspension are applied to the solution in the storage vessel.

The advantage of this embodiment is that cell suspension with high specifications in regard to cell viability, membrane quality and usability of single cells in experiments, such as patch clamp, can be provided for an extended period of time of several hours, which can extend to up to 12 hours. In this way, several experiments can be carried out without the need of providing additional cells within this period of time and without the effort of a continuous cell culture, allowing a dramatic cost reduction and a greater flexibility in experimental handling.

In one embodiment, a flow, preferably a permanent flow that it is strong enough to prevent the accumulation and adhesion of cells, but gentle enough to avoid cell damage, is generated in the storage vessel which accommodates the cells.

The advantage of this embodiment is that a constant flow leads to a constant movement of cells in respect to each other and in respect to the walls of the storage vessel, reducing the probabilities of cells attaching to each other or to the walls of the storage vessel.

In one embodiment, the flow is generated by the design of the interior shape of the storage vessel and constant agitation of the storage vessel and/or ultrasonic treatment.

The interior design of the storage vessel allows modulating the flow of the cell suspension in response to agitation or ultrasonic treatment in such a way, that an even or uneven flow is generated. For example, an even surface can lead to a laminar flow along the surface of the storage vessel, which would counteract cell adhesion to this surface. Alternatively, a surface bearing suitable edges or other obstacles to the liquid flow can lead to the trituration of cell accumulations suspended in the medium, thereby supporting the maintenance of a single cell suspension. The agitation by ultrasonic treatment allows to tightly adjust the flow intensity and, respectively, physical stress imposed on the cells by cavitation, thereby allowing the adjustment of an optimal condition for negative selection of morphologically compromised cells for maintaining a single cell suspension with optimal cell viability and membrane integrity for several hours.

In one embodiment, the storage vessel is provided with a fully or partially concave bottom.

In this embodiment, it is of advantage that a laminar flow along the surface of the interior of the storage vessel is generated, which covers a majority of the surface thereby efficiently counteracting cell adhesion to the surface and cell sedimentation.

In one embodiment, the storage vessel is capable of being sealed, to which end it is preferably sealed with a lid provided with openings.

The sealing of the storage vessel efficiently reduces evaporation of solvent from the storage vessel, ensuring an unchanged concentration of solved buffer components over several hours. This has the advantage of ensuring a substantially unchanged cell viability over several hours and also excludes potential effects on the experiments in which the stored cells are applied. The openings in the lid thereby allow the access to the cell suspension for either retrieving samples from the cell suspension or for adding substances for the preconditioning the cells for their use in experiments.

In one embodiment, the interior of the storage vessel is made of a material, or coated with a material, to which the cells adhere scarcely or not at all.

The advantage of this embodiment is that lower flow velocities are needed in order to inhibit cell adhesion to the surface of the storage vessel, which in turn leads to a reduction of cell damage.

In one embodiment, the cell reservoir is provided with at least one automatic pipetting device in order to cyclically aspirate and expel the solution in the storage vessel, whereby an at least one pipette comprised in the at least one pipetting device is provided with a cell contact area which counteracts cell adhesion, and whereby the at least one pipette is directed in such a way that it expels the aspirated solution onto a curved interior surface of the storage vessel.

The advantage of this method is that the pipetting device by cyclically aspirating and expelling the solution creates a flow, that is persistent but discontinuous in respect to direction, which discontinuity reduces the probability of cell attachment to the walls of the storage vessel still further. Additionally, this device allows the adjustment of the volume surface ratio by adjusting the immersion depth of the pipette mouth into the solution and the volume aspirated by the pipette in the cyclical aspiration and expelling process, which allows a tight adjustment of the oxygenation of the solution present in the storage vessel. Moreover, this device is adjusted to efficiently inhibit foaming of the solution in the storage vessel. Additionally, the passing of the cell suspension through the mouth of the pipette leads to the generation of shearing forces that are preferably adjusted in such a way that they are gentle enough to avoid cell damage, but strong enough to allow the trituration of cell accumulations into single cells.

The invention furthermore relates to a method of subjecting the cells present in single-cell suspension in a storage vessel to intermediate storage in a device in which measures which maintain the single-cell suspension are applied to the solution in the storage vessel.

This method is preferably used together with the novel method of thawing cells, but may also be carried out with cells which have been thawed by another method or with freshly prepared cells, that is to say cells which have not previously undergone cryopreservation.

The invention furthermore relates to cells which, in accordance with the novel method, are provided or subjected to intermediate storage, and a method of carrying out biological assays with biological cells in which the cells, in accordance with the novel method, are provided or subjected to intermediate storage.

Finally, the invention also relates to a device for subjecting, in a storage vessel, cells in single-cell suspension to intermediate storage, which device comprises means for applying the single-cell suspension maintaining measures to the solution in the storage vessel, and which device is preferably arranged to be used in the novel method, and to the use of the device in the novel method.

The invention furthermore relates to a transport vessel with frozen cells which is arranged to be used in the novel method, and to the use of the transport vessel in the novel method.

It is to be understood that the features of the invention which have been mentioned above and which are yet to be illustrated hereinbelow may not only be used in the combination indicated in each case, but also in other combinations or by themselves without thereby departing from the scope of the present invention.

Further features and advantages of the invention can be seen from the following description of preferred embodiments, with reference to the drawing. The figures show:

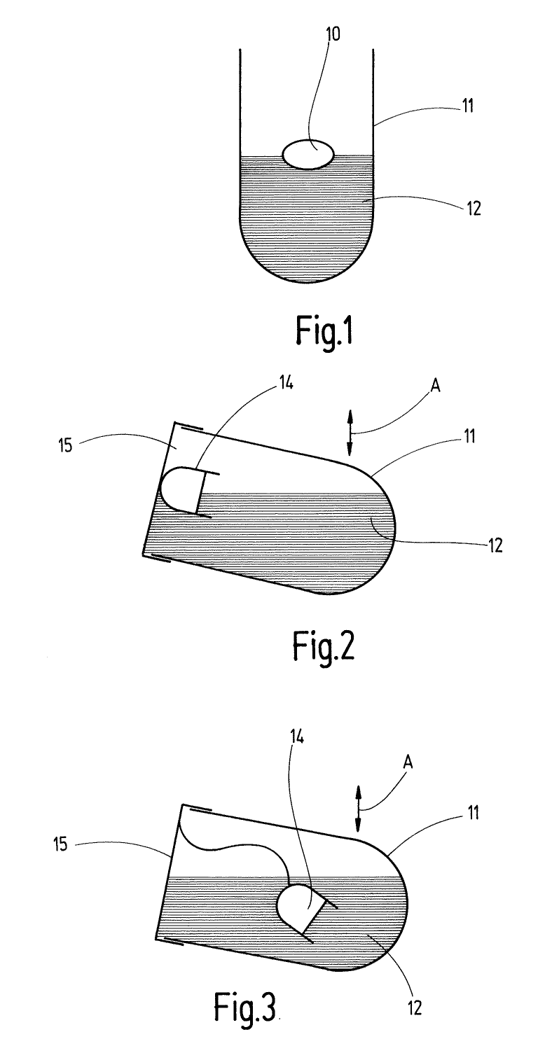

FIG. 1 a thawing procedure in which a pellet with frozen cells is immersed into a thawing vessel with thawing buffer;

FIG. 2 a thawing procedure in which a transport vessel with frozen cells is attached to a lid of a thawing vessel and is brought into contact with thawing buffer by tilting;

FIG. 3 a thawing procedure in which a transport vessel with frozen cells is immersed in such a way that it moves freely in a thawing vessel with thawing buffer;

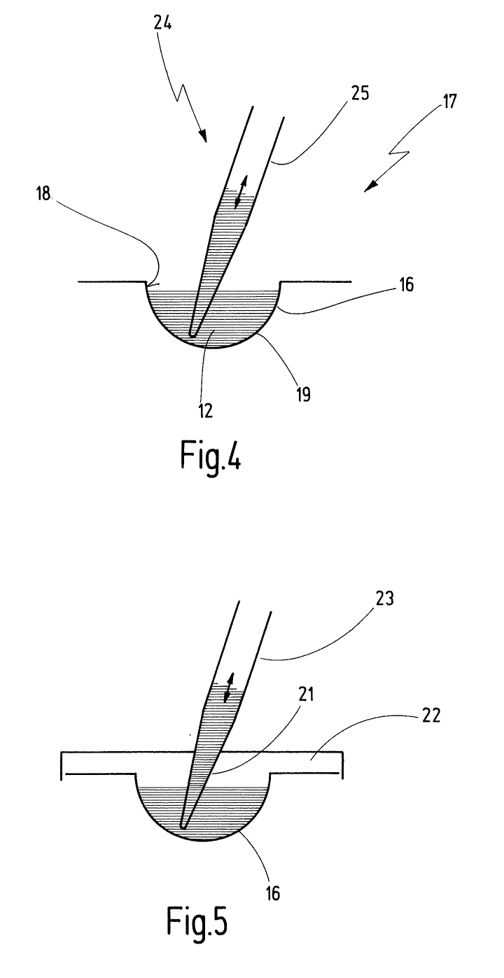

FIG. 4 a diagrammatic representation of a device in which the cells are maintained in single-cell suspension;

FIG. 5 a diagrammatic representation of a storage vessel with lid for use in the device of FIG. 4; and

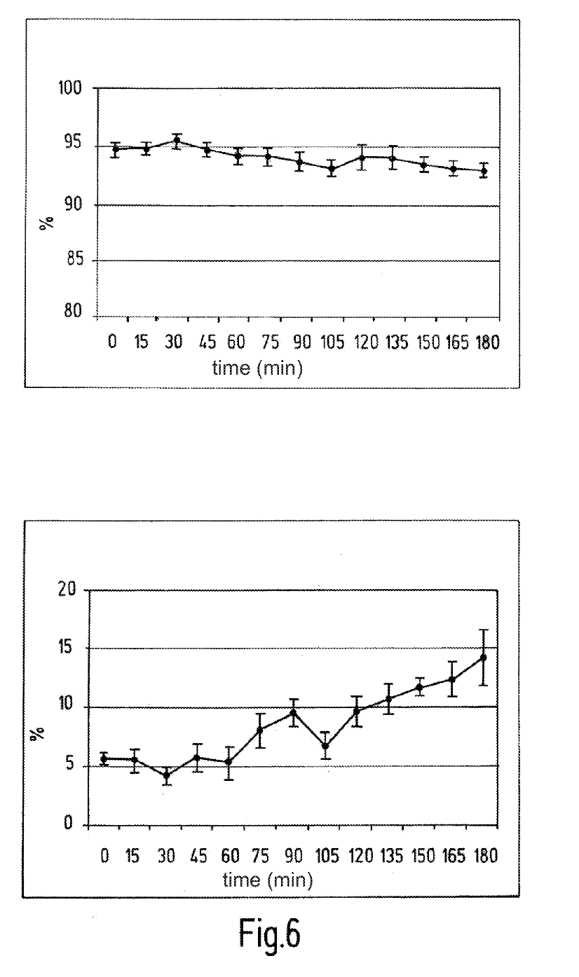

FIG. 6 two test curves in which the diagram at the top shows the course of the vitality of CHO-K1 cells which have been thawed and subjected to intermediate storage according to the novel method, as determined by the trypan blue method; the diagram at the bottom shows the course over time of the percentage of accumulated CHO-K1 cells which have been thawed and subjected to intermediate storage according to the novel method.

The starting point for the novel method are biological cells, for example stably transfected CHO-K1 cells, which express the human ERG (hERG) ion channel. The cells are present as a cell suspension in a storage buffer with a suitable CPA such as DMSO. The cell suspension is then divided into samples of, for example, 0.5 ml with in each case 10×10E6 cells and placed into suitable transport vessels.

These samples together with the transport vessel in question are then frozen by a method known from the prior art as disclosed for example in the documents cited at the outset and stored at low temperature, for example at −80° C. or −196° C. In this state, the samples may be stored over prolonged periods and, in suitable outer containers, also transported over long distances.

At their destination or site of use, the samples are then thawed in one step in accordance with the invention in a manner to be described hereinbelow and then maintained in single-cell suspension over a period of up to several hours. During this time, which may last as long as 12 hours, the samples are used directly in biological assays, either one after the other or several samples in parallel, the biological assays being for example as disclosed in the publications EP 1 218 736 A, EP 0 938 674 A or EP 1 311 655 A mentioned at the outset. As an example, the cells may be used for pharmacological screening, using the patch clamp technique.

In accordance with the invention, the frozen cell suspension samples, for thawing, are brought into direct contact with an excess volume, for example a 10 ml volume, of thawing buffer inside a thawing vessel, which thawing buffer may correspond to the storage buffer, except for the CPA. Thus, in one embodiment 0,5 ml cell suspension is contacted with 10 ml or 12 ml of thawing buffer. A volume ratio of about 1:10 to about 1:40 has proven to be suitable for achieving good cell viability and membrane quality.

For this purpose, the frozen cell pellets 10 are, in a first embodiment, removed from the transport vessel and immersed directly in the thawing buffer 12, which is located in the thawing vessel 11 as shown in FIG. 1.

In another embodiment, an open transport vessel 14 is arranged on the inside of a lid 15 for the thawing vessel 11, which lid is placed on the thawing vessel 11, which thawing vessel 11 is then tilted in order to bring the thawing buffer 12 into contact with the cells 10, as shown in FIG. 2.

In a further embodiment, the open transport vessel 14 is immersed in such a way that it moves freely in the thawing buffer 12, as shown in FIG. 3.

The thawing buffer 12 and the thawing vessel 11 are present at approximately room temperature. The thawing vessel 11 is then maintained at room temperature and—as indicated by the arrow A—shaken carefully, either manually or mechanically. After incubation for approximately 3 minutes at room temperature, the frozen cell suspension is thawed.

As the result of this direct contact with the thawing buffer at room temperature, the frozen cell suspension is thawed in a single step under membrane-conserving conditions, which leads to the membrane quality desired for manual or automated patch clamping.

Firstly, the frozen cell suspension is thawed rapidly and homogeneously without being exposed to strong vibrations. This prevents the formation of intracellular ice crystals caused by recrystallization, and also prevents osmolarity stress as the result of partially thawed media with elevated salt concentration.

Secondly, the CPA is washed out evenly and at room temperature, that is to say at a lower temperature than in the prior art, which prevents osmolarity stress when the CPA is washed out. Finally, a very high dilution—in the present example of 1:20—is achieved very rapidly at room temperature, which leads to a rapid washing out of the CPA from the cells, so that the toxicity of the CPA is neglectable as the result of the low concentration of the CPA—in the present case DMSO—which is established and as the result of the low temperature.

The cells present in the thawed cell suspension are then separated from the supernatant by either centrifugation for 4 minutes at 100 g, or by filtering or sedimentation, the supernatant is discarded and the cell pellet is taken up in the thawing buffer 12 in a storage vessel 16, as shown in FIG. 4.

A high-quality single-cell suspension is thus prepared from the frozen cell suspension in a rapid, simple and inexpensive manner without requiring the effort of a continuous cell culture.

The solution with single-cell suspension prepared this way is then, together with the storage vessel 16, stored for several hours in a device 17, which is shown in FIG. 4 in a highly diagrammatic manner and which is termed cell reservoir, in which device measures which maintain the single-cell suspension are applied to the solution in the storage vessel 16, for example by generating, in the storage vessel 16 in which the cells are located, a constant flow.

As the result, the cells are moved constantly, which prevents their adhesion to the wall 18 of the storage vessel 16 or their accumulation. Moreover, no chemicals such as enzymes need to be added or mechanical means such as beads need to be used in order to maintain the single-cell suspension. Also, the suspension may be kept in the thawing buffer, that is to say the composition of the medium is not changed over all of the procedure. All this leads to a stable suspension with vital cells with intact cell membranes.

The constant flow in this context is preferably adjusted in such a manner that it is strong enough to prevent the accumulation and adhesion of cells, but gentle enough to avoid cell damage.

This constant flow can be achieved by a suitable design of the interior shape of the storage vessel 16 and constant agitation—moving, shaking, tilting and the like—of the storage vessel 16 and/or by ultrasonic treatment.

In one embodiment, the storage vessel 16 is provided with a fully or partially concave bottom 19 in order to make possible a fluid flow in the interior of the storage vessel 16 in a cell-protecting manner.

The storage vessel 16 is furthermore designed in such a way that it is capable of being sealed in order to prevent that fluid evaporates from the storage vessel, thereby altering the osmolarity. On the other hand, the storage vessel 16 must allow the taking up of cells by means of a pipette.

To this end, the embodiment according to FIG. 5 uses a lid 22 provided with openings 21 through which a pipette 23 can be immersed in the solution, the openings, however, being small enough to prevent evaporation.

Finally, the interior of the storage vessel 16 is made of a material, or coated with a material, to which the cells 10 scarcely adhere or not at all. Teflon® has proved to be a suitable material.

In the embodiment according to FIG. 4, the cell reservoir 17 is provided with at least one automatic pipetting device 24 which generates the fluid flow by cyclically aspirating and expelling at least part of the solution in the storage vessel 16.

In one embodiment, the pipette 25 used for this purpose is provided, as the result of a suitable coating, with a cell contact area which counteracts cell adhesion. The pipette 25 is directed in such a way here that it expels the aspirated solution onto the curved interior surface 19 of the storage vessel 16, which leads to a suitable vortexing of the solution and counteracts accumulation and adhesion.

This trituration of the cells 10 across the narrow mouth of the pipette 25 creates mechanical stress and prevents accumulation/clustering of the cells.

Cells which are in a poor state are destroyed as the result of mechanical dissociation, while viable cells remain intact. Thus, the cells are selected over all the time which they spend in the cell reservoir 17.

Experiments carried out by the applicant have shown that the above method allows cryo-preserved biological cells to be transferred into a single-cell suspension which can be used directly in biological assays and to be kept available over a limited period. The cells can be maintained for up to 4 hours in single-cell suspension with an approximately constant cell number and membrane quality. Even after 3 hours, the viability as assessed by the trypan blue test was still substantially over 90% and the number of accumulated cells less than 15%; see FIG. 6.

The frozen cells 10 which have been thawed and subjected to intermediate storage in accordance with the invention were used for determining their electrophysiological properties in a manual and an automated patch-clamp device as they are disclosed in the publications EP 1 218 736 A, EP 0 938 674 A or EP 1 311 655 A mentioned at the outset. The membrane quality and viability of the cells allowed the formation of an outstanding gigaseal and the measurement of currents of the hERG ion channels.

1-28. (canceled)

29. A method for providing suspended cells for carrying out biological assays, comprising the steps of:
a) providing a frozen cell suspension,
b) bringing the frozen cell suspension into direct contact with an excess volume of thawing buffer, and
c) incubating the solution of step b) at room temperature in order to thaw the cell suspension.

30. The method of claim 29, wherein in step b) the thawing buffer is present in at least 10 times the volume of the frozen cells.

31. The method of claim 30, wherein in step b) the thawing buffer is present in the range of about 1:10 to about 1:40.

32. The method of claim 29, wherein in step b) a frozen cell pellet is immersed directly in the thawing buffer which is present in a thawing vessel and at approximately room temperature.

33. The method of claim 31, wherein in step b) a frozen cell pellet is immersed directly in the thawing buffer which is present in a thawing vessel at approximately room temperature.

34. The method of claim 29, wherein in step a) the frozen cells are provided in an open transport vessel and in step b) the transport vessel is immersed directly in the thawing buffer which is present in a thawing vessel at approximately room temperature.

35. The method of claim 31, wherein in step a) the frozen cells are provided in an open transport vessel and in step b) the transport vessel is immersed directly in the thawing buffer which is present in a thawing vessel at approximately room temperature.

36. The method of claim 34, wherein the transport vessel is arranged on an inside of a lid for the thawing vessel, which lid is placed on the thawing vessel in step b), which thawing vessel is then tilted in order to bring the thawing buffer into contact with the cells.

37. The method of claim 35, wherein the transport vessel is arranged on an inside of a lid for the thawing vessel, which lid is placed on the thawing vessel in step b), which thawing vessel is then tilted in order to bring the thawing buffer into contact with the cells.

38. The method of claim 29, wherein the thawing vessel in step c) is shaken by a method selected from the group consisting of manual shaking and mechanical shaking.

39. The method of claim 29, wherein after step c) the cells are separated from the supernatant by a method selected from the group consisting of centrifugation, filtering and sedimentation, the supernatant is then discarded and the cell pellet is taken up once more in the thawing buffer in a storage vessel.

40. The method of claim 39, wherein the storage vessel is subjected to intermediate storage in a device, in which device measures which maintain the single-cell suspension are applied to the solution within the storage vessel.

41. The method of claim 40, wherein a flow is generated in the storage vessel which accommodates the cells.

42. The method of claim 41, wherein the flow is adjusted in such a manner that it is strong enough to prevent the accumulation and adhesion of cells, but gentle enough to avoid cell damage.

43. The method of claim 42, wherein the flow is generated by the design of the interior shape of the storage vessel and by constant agitation of the storage vessel and/or ultrasonic treatment.

44. The method of claim 43, wherein the storage vessel is provided with a concave bottom.

45. The method of claim 39, wherein the storage vessel is sealed with a lid provided with openings.

46. The method of claim 39, wherein the interior of the storage vessel comprises a material, to which the cells adhere scarcely or not at all.

47. The method of claim 39, wherein the cell reservoir is provided with at least one automatic pipetting device for cyclically aspirating and expelling the solution in the storage vessel.

48. The method of claim 47, wherein the pipetting device comprises at least one pipette which, as the result of a suitable coating, is provided with a cell contact area which counteracts cell adhesion.

49. The method of claim 48, wherein the pipette is directed in such a way that it expels the aspirated solution onto a curved interior surface of the storage vessel.

50. A method for providing suspended cells for carrying out biological assays, comprising the steps of:
a) providing a frozen cell suspension,
b) bringing the frozen cell suspension into direct contact with an excess volume of thawing buffer, whereby the thawing buffer is present in the range of about 1:10 to about 1:40, and a frozen cell pellet is immersed directly in the thawing buffer which is present in a thawing vessel at approximately room temperature, and
c) incubating the solution of step b) at room temperature in order to thaw the cell suspension.

51. A method of subjecting, in a storage vessel, cells in single cell suspension to intermediate storage in a device, wherein the cells have been provided according to the method of claim 29, and then are maintained in solution within a storage vessel, and wherein measures which maintain the single-cell suspension are applied to the solution in the storage vessel.

52. A method of subjecting, in a storage vessel, cells in single cell suspension to intermediate storage in a device, wherein the cells have been provided according to the method of claim 50, and then are maintained in solution within a storage vessel, and wherein measures which maintain the single-cell suspension are applied to the solution in the storage vessel.

53. A method for carrying out biological assays on biological cells, wherein the cells are provided by the method of claim 29 or claim 50.

54. A method for carrying out biological assays on biological cells, wherein the cells are subjected to intermediate storage in accordance with the method of claim 51 or claim 52.

55. A device for subjecting, in a storage vessel, cells in single-cell suspension to intermediate storage, which device comprises means for applying single-cell suspension maintaining measures to the solution in the storage vessel.

56. The device of claim 55, which is arranged for carrying out a method of subjecting, in said storage vessel, cells in single cell suspension to intermediate storage in said device, wherein the cells have been provided from a frozen cell suspension that has been brought into direct contact with an excess volume of thawing buffer, whereby the so obtained solution has been incubated at room temperature in order to thaw the cell suspension,
and wherein the cells then are maintained in solution within a storage vessel, and wherein measures which maintain the single-cell suspension are applied to the solution in the storage vessel.
